(19) United States
(12) Patent Application Publication
(10) Pub. No.: US 2008/0248486 A1
(43) Pub. Date: Oct. 9, 2008

(54) Type Pept1 Protein Assay

(75) Inventors: Natalie Watzke, Weinheim (DE); Maarten Ruitenberg, Eppstein (DE); Wolfgang Dorner, Hochheim (DE); Renate Gauss, Frankfurt (DE); Bela Kelety, Frankfurt (DE); Joanna Tobien, Glienicke/Nordbahn (DE)

Correspondence Address: D. PETER HOCHBERG CO. L.P.A., 1940 EAST 6TH STREET, CLEVELAND, OH, 44114, US

(73) Assignee: Iongate Biosciences GmbH, Frankfurt (DE)

(21) Appl. No.: 11/664,541

(22) PCT Filed: Sep. 30, 2005
(86) PCT No.: PCT/EP05/10601

§ 371 Date: Mar. 24, 2008

(30) Foreign Application Priority Data
Oct. 1, 2004 (DE) 10 2004 048 391.4

(51) Int. Cl. G01N 33/53; A61K 38/54

(52) U.S. Cl. 435/7.1; 514/2

(57) ABSTRACT

The present invention relates to a type PepT1 protein assay and in particular a process for identifying a substrate and/or a modulator of the PepT1 protein.

### CROSS-REFERENCE TO RELATED APPLICATIONS

This application is a National Stage application of International Application No. PCT/EP2005/010601, filed on Sep. 30, 2005, which claims priority of German application No. 10 2004 048 391.4, filed on Oct. 1, 2004.

### BACKGROUND OF THE INVENTION

1. Field of the Invention

The invention relates to a type PepT1 protein assay and in particular a process for identifying a substrate and/or modulators of a type PepT1 protein.

The invention relates moreover to an active principle complex which has been identified according to the process of the invention, a process for the production of a drug which is a substrate for the type PepT1 protein or has been made into such a substrate by conjugation with a substrate for a type PepT1 protein, a test kit for carrying out the process according to the invention, a screening process and the use of the process according to the invention, the test kit according to the invention and the drug.

2. Description of the Prior Art

The extremely rapid progress in the development of pharmaceutical active principles is causing a rapid increase in the number of potentially available therapeutic agents. However, a serious problem is still very frequently posed by formulating the corresponding agents in such a way that they exhibit good oral bioavailability, i.e. they are absorbed sufficiently well by the body on oral intake to ensure an optimum effectiveness and/or tolerance.

Natural transport proteins play an important part in absorbing different molecules e.g., from the intestines.

Polar or hydrophilic compounds are usually absorbed only poorly in the intestine since their transportation via the cell membrane is unfavourable from the energy point of view and requires energy expenditure. Amino acids, dipeptides and tripeptides, monosaccharides, vitamin nucleosides etc. are such polar compounds, the absorption of which, however, is essential for the organism. There are usually specific transport systems for such substances.

In mammals, two peptide transporters, PepT1 and PepT2, have been detected so far. PepT1 is expressed in the small intestine, the kidney, the bile duct and the pancreas whereas PepT2 is expressed in the kidney, the central nervous system (CNS), the peripheral nervous system (PNS), the lung, the mammary gland, the spleen, the large intestine and the pancreas (survey by Rubio-Aliaga & Daniel 2002).

At the beginning of the 1960s, evidence of the active absorption of Gly-Gly in the intestinal epithelial cells was successfully provided (Newey & Smyth 1962). The carrier is dependent on an H+ gradient facing cell inwards (Ganapathy & Leibach 1985). It is stimulated by the membrane potential, saturable and electrogenic. By expression cloning in oocytes of Xenopus laevis, it was possible in the mid 1990s to clarify the primary structure initially of the intestinal peptide transporter (PepT1) and shortly afterwards of the renal isoform (PepT2) (Fei et al. 1994; Boll et al. 1996).

From the large number of its structurally diverse natural substrates (8400 dipeptides or tripeptides) it is evident that the substrate specificity of the peptide transporters is not strongly developed.

Thus, some investigations showed as early as at the beginning of the 1970s that the transporter recognises, apart from its natural substrates, also lactam antibiotics which possess a tripeptide-type structure (Quay 1972). Thus, the peptide transport system becomes interesting to the pharmaceutical industry since, the oral application of peptide pharmaceuticals can be made possible by means of te peptide transport system.

From the investigations carried out so far into the substrate specificity of the intestinal H+/peptide symporter it was possible to derive a few and partially contradictory statements regarding the factors for an optimum recognition by the transporter.

The peptide bond and a free N-terminus and C-terminus were regarded as necessary for a long time for an optimum interaction between the substrate and the transporter. More recent findings provide cause for revising this view. It has, for example, been possible to show that the peptide transporter recognises a ketomethylene group (Dóring et al. 1998a) or an ester bond (Ganapathy, M. E. et al. 1998) instead of a peptide bond of similar affinity.

In the literature on inhibitors of the peptide transporter, either contradictory results have so far been published (Taub et al. 1997; Abe et. al. 1999; Yang et al. 2002) or the inhibitor described exhibited only a slight affinity to the transporter (4-aminomethyl benzoic acid: Meredith et al. 1998).

In addition, voluminous blocking groups are partly tolerated by the transporter with a relatively slight affinity reduction. Apart from identifying inhibitors, the possibility also arises of developing prodrugs by coupling orally non-available medicines, for example, to the side chain of the dipeptide.

Since PepT1, but not PepT2, is expressed in the intestine, efforts nowadays have been aimed at improving the oral availability of medicines by searching for pharmacological agents which are substrates for PepT1 or can be modified in such a way that they can be recognised and transported by PepT1.

Various processes and measuring devices are known in the state of the art by which properties of certain active principles can be analysed quantitatively and qualitatively and described.

In this respect, it is desirable to apply given and known active principles on the active site complex, e.g. on PepT1 protein on a cell, a tissue or such like in order to influence and/or investigate the method of operation of this active site.

The usual experiments on the organism as a whole are often questionable because of the complexity of the organism and also as a result of ethical and moral circumstances and the results thus obtained consequently have only a limited validity.

Consequently, processes and devices have been developed by which an isolated investigation of the method of action of the active principles to be tested on isolated centres of action or an investigation of the centres of action themselves is possible. In this respect, certain tissues, isolated cells and/or other biological units, e.g. proteins or such like, are made accessible to investigation in an isolated manner. Techniques are known, for example, which operate by using dyes or radioactive substances and in which changing transport and/or bonding specificities are exploited and represented. Where this procedure is possible at all, it has the disadvantage, however, that it has frequently only a slight resolution capacity and a high error susceptibility.

Frequently, such methods are also relatively insensitive.

Electro-physiological methods are also known, such as the so-called patch clamp technique or voltage clamp technique, in which cells, for example, in the isolated state are accessible to electro-physiological investigation. In the case of this procedure, native cells are subjected to different conditions and certain electrical activities which are mediated via the cell membrane by proteins, protein complexes or such like contained therein, are measured.

In spite of the high accuracy thus achievable, these know electro-physiological methods have the disadvantage that, as a result of their high measurement technology requirements and their susceptibility to interference, they are unsuitable for rapid, automatable and/or wide-ranging use and, as a result of the usually native environment of the objects to be investigated, exhibit certain problems regarding the discrimination between undesirable parts of signals. In addition, these methods are highly cost intensive.

A particularly advantageous process for measuring electro-chemical processes on e.g. membrane fragments is described in WO 02/1074983 to the full content of which reference is made here for the purpose of the present application. The use of the process disclosed therein for testing operations on type PepT1 proteins is not disclosed.

### SUMMARY OF THE INVENTION

The invention is consequently based on the object of creating a type PepT1 protein assay in which a rapid test of the active principle is possible in a particularly simple and yet reliable manner, in particular in a large scale operation, at acceptable costs.

The present invention creates, according to an embodiment, a process for identifying an active principle complex which modifies an enzymatic property of an active site complex containing a type of an EACC1PepT1 protein.

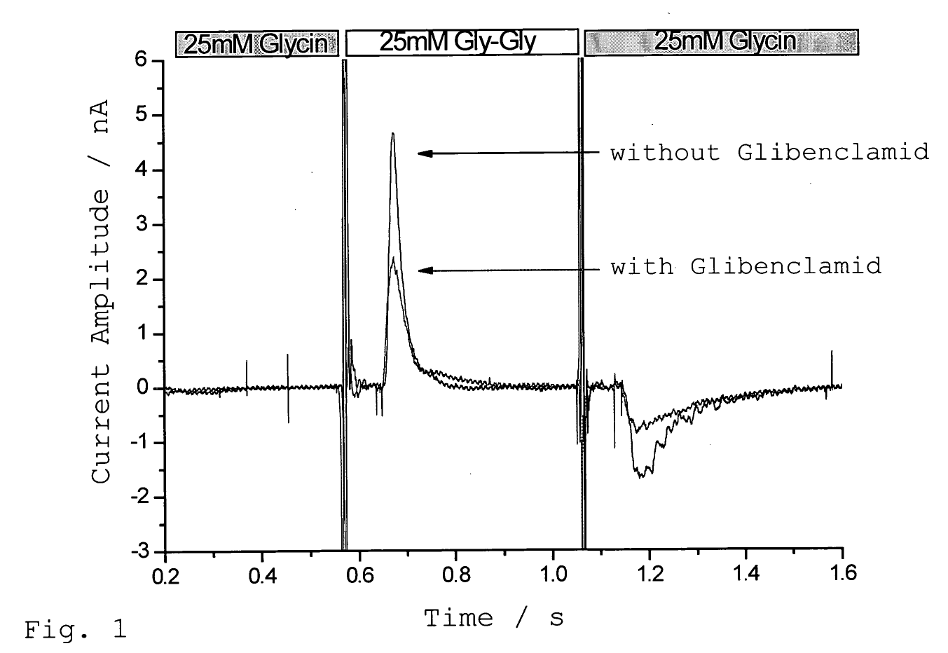

FIG. 1 shows diagrammatically in the form of a graph the transport stream I(t) produced by the PepT1 protein as an action measurable elecrically by the biosensor electrode.

### DESCRIPTION OF EMBODIMENTS

In the transport cycle of PepT1, 1 (according to the latest findings possibly 2) proton(s) is/are displaced via the membrane together with 1 substrate molecule, naturally 1 small peptide in particular 1 dipeptide or tripeptide normally present in the intestine.

The invention is, among other things, based on the idea of measuring the arising charge transfer—i.e. the ion transport of protons and/or of charged substrates such as e.g. dipeptides or tripeptides or of electrically non-charged substrates—directly by binding enzyme preparations to a suitable surface which is integrated into a continuous flow system and/or in the case of which a substrate jump can be carried out, as electrical current or as electrical potential change and/or to investigate the influence of different substrates on the electrical measuring parameters of electric current or potential.

The invention relates according to another embodiment to a process for identifying potential substrates and/or inhibitors/activators of a type PepT1 protein which modifies an enzymatic property and/or the transport behaviour of the type PepT1 protein or a part thereof. The process according to the invention has the following steps:
(a) Providing a plurality of primary carriers which, containing the active site complex, comprises the type PepT1 protein, in particular in a plurality in the region of a membrane of the primary carrier concerned,
(b) Attaching or bringing into contact the primary carrier to or within the surface area of an isolation area of a biosensor electrode serving as secondary carrier in the attachment buffer, the secondary carrier being or becoming electrically isolated preferably vis-á-vis the attachment buffer and vis-á-vis the primary carriers by means of the isolation area,
(c) Providing at least one potential active principle complex,
(d) Bringing into contact and in particular bringing into interaction of the potential active substance complex with the active site complex of the primary carrier or parts thereof and
(e) Determining the qualitative and/or quantitative influence of the potential active principle complex or a part thereof on enzymatic properties of the active site complex or a part thereof by detecting an electrical action of the or on the active site complex or a part thereof or a change-of this electrical action via the biosensor electrode as secondary carrier,

One or several of the following sub-steps being carried out in at least one of the process steps (c), (d) and (e):
(f) Introducing the secondary carrier with the primary carriers into a second non-activating solution preferably containing a compensator and, if necessary, detecting an electrical action according to or according to the meaning of, process step (e),
(g) Introducing the secondary carrier with the primary carriers into a third or activating solution and detecting an electrical action according to or according to the meaning of process step (e), the third or activating solution corresponding to the second or non-activating solution but additionally containing a substrate of the type PepT1 protein and, if necessary, the compensator.

For this, a solution with 140 mmol/1 KCl, 2 mmol/1 MgCl₂, 30 mmol/1 Mes, pH 6.0 or Hepes pH 7.0 and 25 mmol/1 glycine can be used as non-activating solution.

Moreover, a solution with 140 mmol/1 KCl, 2 mmol/1 MgCl₂, 30 mmol/1 Mes, pH 6.0 or Hepes pH 7.0 and 25 mmol/1 Gly-Gly can be used as activating solution.

The steps according to the invention (f) and (g) are—if necessary with repetitions—preferably carried out in the sequence indicated.

It may be particularly advantageous if a step (f′) is carried out once before step (f), incubation being carried out with an attachment buffer rather than with a non-activating solution, the attachment buffer corresponding to the non-activating solution, though the compensator not being contained in the solution.

If it is to be checked whether a potential active principle is being transported, the potential active principle should be present only in the activating solution but not in the non-activating solution.

If it is to be checked whether a potential active principle activates/inhibits the type PepT1 protein, the potential active principle can be present in all the media.

Preferably, a monomer or an oligomer of a PepT1 protein is used as active site complex or a part thereof.

According to the invention, the term “type PepT1 protein” means that the protein has the typical characteristics of the PepT1 protein. In particular, it is understood to mean proteins which resemble or correspond regarding their transport and substrate recognition properties and their sensitivity vis-á-vis inhibitors and activators to PepT1 protein or the renal isoform PepT2. Both isoforms are simple to use in the present test system.

It is, moreover, anticipated that an active site complex or a part thereof is used which is based on a type PepT1 protein originating from a tissue of a mammal, e.g. for the small intestine, kidney, bile duct or pancreas or derived therefrom.

In particular, the type PepT1 protein originates from mammal cell lines and is present in the cloned form.

Advantageously, it is provided for the active site complex (containing the type PepT1 protein) to originate from the organism of pig, mouse, sheep or man or of being genetically derived therefrom.

For the purposes of the present invention, the term “enzymatic property” of type PepT1 protein always means the transport behaviour, e.g. the peptide transport and/or proton transport mediated by these proteins and consequently also relates to the influence, discussed above, of the protein on the bioavailability of potential active principles. This means that where the modification of the enzymatic properties of the protein is involved, investigations according to the invention are included which are to show whether a certain substance is transported by type PepT1 protein and/or whether a certain substance modifies the transport properties of the type PepT1 protein, i.e. is a modulator according to the invention of the type PepT1 protein.

For the purposes of the present invention, the term active principle complex means either a potential substrate whose transport is to be investigated via the type PepT1 protein or a so-called modulator which inhibits or activates the transport of previously determined substances, for example.

According to the invention, it is possible to carry out process steps (c) and/or (d), if necessary also (f), (f′) and/or (g) by
admixing or injecting the active principle complex, a part and/or a precursor thereof,
replacing or admixing the measuring solution or a part thereof and/or
chemically or physically converting or reacting the measuring solution or a part thereof or the active principle, a part and/or a precursor thereof.

This means that, on the one hand, the active principle complex or a part thereof can be transported directly by injecting or admixing into the measuring solution to the site of the active site complex. However, such a process is particularly simple if the measuring solution concerned is simply replaced, for example in a continuous manner. Moreover, it is conceivable that the active principle is liberated in the measuring solution only by a chemical or physical conversion or reaction. This can take place by supplying radiation, for example.

The qualitative influence and/or the quantitative influence of the potential active principle or active principle complex is determined according to the invention by detecting an electrical action which is mediated by the active site complex or a part thereof and in particular by the type PepT1 protein.

In particular, this electrical action is determined with or without a potential active principle and it is examined by comparing the two series of measurements whether the potential active principle influences the enzymatic properties of the type PepT1 protein.

As described in WO02/074983, the primary carriers are brought into contact with the active site complexes on a secondary carrier, namely the biosensor electrode, and in particular with its isolation area and, in particular, attached thereon.

Numerous possibilities are available for designing the biosensor electrode as secondary carrier.

However, it is particularly preferred if a biosensor electrode is used as secondary carrier in the case of which an electrically conductive and solid-type electrode area with at least one electrode is provided which is electrically insulated vis-á-vis the measuring solution and vis-á-vis the primary carriers by providing an insulation area in the form of a solid-supported membrane which is formed as a layer structure of a bottom layer of an organic thiocompound as lowest layer facing the electrode and an upper layer of an amphilic organic compound.

In a preferred embodiment of the process according to the invention, it is provided that a biosensor electrode is used as secondary carrier in which an electrode of gold is provided in the electrode area, with a mono-layer of a long chain alkane thiol as sublayer thereon and a mono-layer of a lipid as upper layer thereon.

With respect to the primary carriers which are to contain the active site complex and in particular the type PepT1 protein in the area of their membrane, different possibilities arise, it being possible to take the aims of the process into account in each case.

According to a particularly advantageous embodiment of the process according to the invention, it is, for example, possible to use, as primary carrier, a eukaryotic cell, a prokaryotic cell, in particular an oocyte, a bacterium, a virus, an organelle or components thereof, in particular membrane fragments or composites thereof in the native form and/or in an altered form, in particular in the purified and/or modified form.

It is also possible to use as primary carrier, a vesicle, a liposome or a micellar structure. The membrane fragments can also be attached in a planar manner i.e. as non-spherical structure.

The process according to the invention is particularly advantageous if the sensor arrangement of biosensor electrode as secondary carrier and primary carrier attached thereto has the measuring solution streaming around or to it in a measuring chamber, measuring area or measuring vessel. In this way, it is possible, by changing the measuring solution, to easily implement a concentration jump, for example, with regard to the active principle complex or a part thereof. It is also possible to adjust, by such measuring within the framework of a flow system, the test conditions according to the invention advantageously, easily and reliably with a high time resolution. The process according to the invention can also be carried out advantageously by an automated pipetting device.

The process according to the invention is particularly economical and easily accessible to statistical evaluation if, in sequence, a multiplicity of tests is carried out, in particular by exchanging the measuring solution in sequence, if necessary with washing or rinsing of the measuring chamber in between.

It is important in the case of the present process according to the invention, to compare the action of the active site complex in the presence of the active principle complex with a situation in which the potential active principle complex is not present. This can be effected, for example, by changing the non-activating to the activating solution in the presence or absence of the potential active principle complex and comparing the measured values obtained.

Potential modulators to be tested are in particular preferably monoclonal antibodies, antibody fragments, polyclonal antibodies and peptides. However, other substances can also be added of which it is thought that they are capable of developing, a corresponding effect. These substances are preferably administered in the dissolved form.

Particularly preferred substances which can be investigated as possible potential substrates and/or modulators in the test system according to the invention are low-molecular compounds. Such compounds frequently have no or only slight secondary effects if they are used as active principle in a pharmaceutical composition. A further advantage of such substances is the possibility of oral administration.

Examples for this are cyclic pentapeptides such as those described by Haubner et. Al., J. Am. Chem. Soc. 1996, 118, 7641-7472. According to the invention, small peptides, amino acids and amino acid analogues, steroids, nucleotides and other organo-chemical substances with a molecular weight of ≦5000, preferably ≦3000 and particularly preferably ≦2000 are counted as low molecular substances.

The active principle complex can be added both in the attachment buffer, in the non-activating as well as in the activating measuring solution, or liberated therein.

An incubation of the active site complex or the primary carrier carrying it with the active principle complex is also conceivable as intermediate step.

Moreover, it is conceivable that a wash step is carried out between steps (f) and (g) by introducing the secondary carrier with the primary carriers into a (wash) solution which is preferably identical to the attachment buffer.

According to the invention, the aqueous measuring solutions and in particular aqueous electrolyte solutions are used as measuring solution which can be referred to as attachment buffer, non-activating and activating solution.

All the measuring solutions used contain an agent stabilising the pH which is known to the expert, preferably selected from the list of: MOPS, HEPES, MES, Tris, PIPES etc. such as those published e.g. in “Buffers, Calbiochem” but also those which can be easily found in further publications and textbooks of biochemistry and/or organic chemistry. According to the invention, these agents known as such should have a stabilising effect in the region of pH 6-8, preferably at approximately 7. The choice of the suitable pH range and agent may depend on the substrate (active principle complex) and can be easily determined by the expert by routine experiments.

The attachment buffer comprises at least one cation species, preferably selected from the list consisting of K⁺, Ca⁺⁺, Na⁺, Li⁺, Mg⁺⁺, choline⁺, Rb⁺, Sr⁺⁺ in a concentration which is preferably approximately as high as the cation concentration in the non-activating and in the activating solution. It is, for example, possible for this concentration to be between 1 μmol/l and 1 mol/l, preferably between 10 and 200 mmol/l, particularly preferably between 120-160 mmol/l.

The non-activating solution also contains at least one cation species in a concentration which is preferably approximately as high as the cation concentration in the attachment buffer.

Preferably, a cation in the list described for the attachment buffers is involved, it preferably corresponds to the cation or the cations of the attachment buffer.

Moreover, the non-activating solution may, if necessary, comprise a compensator. This compensator has the purpose that, on changing over from the non-activating solution to the activating solution e.g. by changing the ionic strength, no falsely positive signal which is independent of the active site is detected.

Preferably, the compensator is an amino acid and particularly preferably the amino acid glycine if the dipeptide Gly-Gly is used as substrate in the activating solution during inhibition measurements.

The type of compensator used depends in particular on the type of substrate used.

If a dipeptide or tripeptide is used as substrate, an amino acid with a pK value is preferably used as compensator which is approximately identical to the pᵢ value of the dipeptide or tripeptide.

In the case of investigations regarding the potential transport of target molecules, an acid or a base is used as compensator which has a pK value which corresponds approximately to the target molecule investigated.

The concentration of the compensator generally depends on the concentration of the substrate and is generally between 0 and 100 mmol/l. In principle, the concentration of the compensator is varied until an electrical action independent of the active site no longer arises as a result of the change over from non-activating to activating solution.

The activating solution may contain the compensator irrespective of whether this is contained in the non-activating solution.

The activating solution also contains at least one cation species in a concentration which is preferably approximately as high as the cation concentration in the attachment buffer.

Preferably, it consists of a cation contained in the list described for the attachment buffer, it preferably corresponds to the cation or the cations of the attachment buffer. Moreover, it contains a substrate of the type PepT1 protein, preferably a dipeptide or tripeptide, particularly preferably Gly-Gly for inhibition or activation measurements.

Further possible substrates are, for example, monosaccharides, vitamins, nucleosides and beta-lactam antibiotics and compounds with a similar structure. In particular, peptide-type compounds are involved. The concentration may vary between >0 and 1 M, depending on the application.

The corresponding concentrations can be easily determined by the expert.

In a further aspect of the present invention, the compensator may also be present in the activating solution if, in this way, a better signal background ratio is obtained, i.e. the absence of falsely positive signals independent of the active site can be achieved on changing over from the non-activating solution to the activating solution.

Cations suitable according to the invention are all cations which (a) do not inhibit PepT1 and (b) do not trigger measuring artefacts. It is known, among other things, of Zn and Cu that they inhibit PepT1.

According to a further aspect, the present invention creates an active principle or an active principle complex which modifies an enzymatic property of an active site complex containing a type of a PepT1 protein, or a part thereof, and which is identified, will be so or has been so for identifying an active principle complex according to the process of the invention.

Moreover, the present invention creates a process for producing a drug using the steps of:
Identifying an active principle and/or an active principle complex which modifies an—if necessary specific—enzymatic property of an active principle complex containing a type of a PepT1 protein, or a part thereof, or a multiplicity of properties in a suitable manner, namely by means of the process according to the invention,
Producing and/or isolating the active principle, the active principle complex, a part and/or a derivative thereof,
If necessary purifying the active principle, active principle complex, part and/or derivative,
If necessary mixing and/or portioning the active principle, active principle complex, part and/or derivative with a pharmaceutically compatible carrier substance.

Moreover, the present invention creates a test kit for carrying out the process according to the invention for identifying an active substance complex. This test kit exhibits:
at least one primary carrier with the type PepT1 protein,
a measuring area,
if necessary the attachment buffer according to the invention, the non-activating solution and the activating solution and
moreover, if necessary, at least one potential active principle complex.

According to the invention, a screening process for identifying is also created, namely for identifying:
an unknown active principle, active principle complex, a part and/or derivate thereof,
the presence of an unknown active principle, active principle complex, part and/or derivative thereof,
the presence of a known active principle, active principle complex, part and/or derivative thereof,
the concentration of an unknown active principle, active principle complex, part and/or derivative thereof,
the concentration of a known active principle, active principle complex, part and/or derivative thereof,

According to a further aspect of the present invention, the process according to the invention is used to identify an active principle complex in order to find inhibitors, activators, partial or temporary inhibitors or modulators with an enzymatic property of an active site complex containing a type PepT1 protein.

Moreover, the test kit according to the invention is used according to the invention to find modulators, i.e. inhibitors or activators, e.g. partial or temporary inhibitors of an active site complex containing a type of a PepT1 protein.

Preferably, the active site complex contains the PepT1 protein.

Moreover, the drug is used according to the invention to inhibit, partially or temporarily inhibit, activate or otherwise modulate an active principle complex containing a type PepT1 protein.

Based on the following remarks, the above and further aspects of the present invention will be elucidated using other words:

Moreover, a measuring solution, which is e.g. aqueous, is provided in which the primary carrier and the secondary carrier are arranged. The electrode area is preferably largely electrically insulated vis-á-vis the measuring solution (activating and/or non-activating solution), the primary carriers and vis-á-vis the biological units.

The electrode area is equipped e.g. with at least one electrode. On the one hand, this can be formed itself as a mechanically stable material area, in particular as a plate, a wire and/or such like.

A particularly simple arrangement of the sensor electrode device is obtained if the electrode is formed as essentially material layer deposited on the surface of the carrier. It may consist of a vapour deposited or sputter deposited material layer. The material layer for forming the electrode preferably has a layer thickness of approximately 10 to 200 nm.

The type PepT1 protein may be provided essentially in the native form and/or in an altered, in particular purified, microbiologically and/or molecular-biologically modified form. On the one hand, certain native properties can be tested and pharmacologically investigated with it. On the other hand, molecular biological or gene technologically initiated modifications are possible for analysing certain aspects, e.g. the transport or the pharmacological method of action of an active principle.

It is particularly advantageous that primary carriers of an essentially uniform type of primary carrier are provided. This is important with respect to a possible unambiguous result and analysis of an active principle test and relates to the geometrical, physical, chemical, biological and molecular-biological properties of the primary carrier.

The same applies also to the active site complexes provided in the primary carrier and the type PepT1 protein. In this case, the active site complexes and the type PepT1 protein of an essentially uniform type are provided in each case, in particular with respect to their geometrical, physical, chemical, biological and molecular-biological properties. In addition, these biological units should advantageously be approximately uniform with respect to their orientation and/or with respect to their activatibility vis-á-vis the primary carrier concerned.

As has already been detailed, the invention is, among other things, based on the object of making available to investigation the effective of substances on the operation of Type PepT1 proteins. Substances which modulate the effect of this transport protein are of commercial interest as potential neuroprotective active principles. Substrates of the protein which are transported by it, too, would possibly be important new molecules.

The procedure according to the invention provides the following advantages:

Measuring the transport activity and/or the transport properties according to the process proposed according to the invention requires no mediators or labelled substrates. An individual measurement takes only a few seconds. The measurement is sensitive vis-á-vis all substrates. Insofar as a substrate does not irreversibly inhibit the reaction, sensors loaded with an enzyme are capable of carrying out several measurements. The substrates need not be present in the chemically modified form since the current response and potential response of the protein is induced by a rapid solution change.

In addition, a higher rate of throughput is possible in contrast to the patch clamp technique.

### EXAMPLE 1

### Production of the EAAC1 Sensor Chip

10 μl of a membrane suspension (protein concentration approximately 2-3 mg mLml⁻¹) of a CHO plasma membrane preparation was applied onto a pretreated sensor chip with a round gold electrode (diameter 3 mm, obtainable from IonGate Biosciences GmbH, Frankfurt am Main) and incubated in the refrigerator (at <8° C.) over night.

The capacity of the protein-loaded membranes was approximately 1000 nF cm⁻², the conductivity Gₗₒ approximately 10 nS cm⁻².

### EXAMPLE 2

### Activity Measurement without Initiator

The flow cell of a SurfE²R system (IonGate Biosciences GmbH, Frankfurt am Main) with an integrated protein-loaded sensor chip was first rinsed with attachment buffer (compare above). For the actual measurement, a changeover was effected by means of an electro-mechanical 3/2-way valve between the non-activated solution (140 mmol/l KCl, 2 mmol/l MgCl₂, 30 mmol/l MES pH 6.0 or Hepes pH 7.0+25 mmol/l glycine) and the activating solution (140 mmol/l KCl, 2 mmol/l MgCl₂, 30 mmol/l MES pH 6.0 or Hepes pH 7.0+25 mmol/l substrate e.g. Gly-Gly).

### EXAMPLE 3

### Activity Measurement with Glibenclamide

Results compare FIG. 1

The measurements were carried out as above, although both solutions contained activating and non-activating solution and additionally 100 μM glibenclamide. Glibenclamide is an inhibitor for PepT1 (IC₅₀ in oocytes approximately 100 μM). The inhibition of the current amplitude by glibenclamide proves that the detected signal is indeed a specific PepT1 signal. A complete inhibition with glibenclamide is not possible as a result of the poor solubility of glibenclamide.

FIG. 1 shows diagrammatically in the form of a graph the transport stream I(t) caused by the PepT1 protein as electrical action measurable by the biosensor electrode.

The basic advantages of the present invention are the high mechanical stability of the sensor arrangement provided and simultaneously the high level of usability, the simple handleability and the low susceptibility to problems. Regarding the use of the sensor arrangement, a long useful life, a high level of reliability, a low susceptibility to problems as well as in particular a notably increased test throughput compared with conventional processes are obtained within the framework of pharmacological active principle tests, as a result of which it was possible to develop and execute corresponding test processes in a cost effective manner.

In the sense of the present invention an active principle and an active principle complex may also be referred to as an agent, as an active agent, as an active component, as an active ingredient, and as an active substance and as an agent complex, as an active agent complex, as an active component complex, as an active ingredient complex, and as an active substance complex, respectively.

What has been described above are preferred aspects of the present invention. It is of course not possible to describe every conceivable combination of components or methodologies for purposes of describing the present invention, but one of the ordinary skill in the art will recognize that many further combinations and permutations of the present invention are possible. Accordingly, the present invention is intended to embrace all such alterations, combinations, modifications, and avariations that fall within the spirit and scope of the appended claims.

1-21. (canceled)

22. A process for identifying an active principle complex which modifies an enzymatic property of an active site complex containing a type of an PepT1 protein, said process comprising the steps of:
(a) providing a plurality of primary carriers, each primary carrier having a membrane region and containing an active site complex containing a plurality of the type PepT1 protein in said membrane region of the primary carrier concerned;
(b) attaching and bringing into contact each primary carrier of said plurality of primary carriers to or within the surface area of an isolation area of a biosensor electrode, said biosensor electrode being a secondary carrier in a measuring or attachment buffer medium, the secondary carrier being or becoming mechanically and electrically isolated vis-á-vis the measuring or attachment buffer medium and vis-á-vis the plurality of primary carriers by the isolation area;
(c) providing at least one potential active principle complex;
(d) bringing into contact and bringing into interaction of the potential active principle complex with the active site complex of the plurality of primary carriers or parts of said plurality of primary carriers; and
(e) determining the qualitative and/or quantitative influence of the potential active principle complex, or a part thereof, on enzymatic properties of the active site complex, or a part thereof, by detecting an electrical action of the active site complex, or a part thereof, or a change of the electrical action via the biosensor electrode as the secondary carrier.

23. The process according to claim 22, further comprising at least one of the following sub-steps carried out in at least one of the process steps (c), (d) and (e):
(f) introducing the secondary carrier with the plurality of primary carriers into a second or non-activating solution; and
(g) introducing the secondary carrier with the plurality of primary carriers into a third or activating solution and detecting an electrical action according to said process step (e), wherein the third or activating solution corresponds to the second or non-activating solution but additionally contains a substrate of the type PepT1 protein.

24. The process according to claim 23, wherein said non-activating solution is a solution comprising 140 mmol/l KCl, 2 mmol/l MgCl₂, 30 mmol/l Mes, pH 6.0 or Hepes pH 7.0 and 25 mmol/l glycine.

25. The process according to claim 23, wherein said activating solution is a solution comprising 140 mmol/l KCl, 2 mmol/l MgCl₂, 30 mmol/l Mes, pH 6.0 or Hepes pH 7.0 and 25 mmol/l Gly-Gly.

26. A process for identifying an active principle complex which modifies an enzymatic property of an active site complex containing a type of an PepT1 protein, said process comprising the steps of:
(a) providing a plurality of primary carriers, each primary carrier having a membrane region and containing an active site complex containing a plurality of the type PepT1 protein in the membrane region of the primary carrier concerned;
(b) attaching and bringing into contact each primary carrier of said plurality of primary carriers to or within the surface area of an isolation area of a biosensor electrode, said biosensor electrode being a secondary carrier in a measuring or attachment buffer medium, the secondary carrier being or becoming mechanically and electrically isolated vis-á-vis the measuring or attachment buffer medium and vis-á-vis the plurality of primary carriers by the isolation area;
(c) providing at least one potential active principle complex;
(d) bringing into contact and bringing into interaction of the potential active principle complex with the active site complex of the plurality of primary carriers, or parts of said plurality of carriers; and
(e) determining the qualitative and/or quantitative influence of the potential active principle complex or a part thereof on enzymatic properties of the active site complex or a part thereof by detecting an electrical action of the active site complex or a part thereof or a change of the electrical action via the biosensor electrode as the secondary carrier, wherein at least one of the following sub-steps is carried out in at least one of the process steps (c), (d) and (e):
(f) introducing the secondary carrier with the plurality of primary carriers into a second or non-activating solution; and
(g) introducing the secondary carrier with the plurality of primary carriers into a third or activating solution and detecting an electrical action according to process step (e), wherein the third or activating solution corresponds to the second or non-activating solution but additionally contains a substrate of the type PepT1 protein, wherein said non-activating solution is a solution comprising 140 mmol/l KCl, 2 mmol/l MgCl₂, 30 mmol/l Mes, pH 6.0 or Hepes pH 7.0 and 25 mmol/l glycine, and wherein said activating solution is a solution comprising 140 mmol/l KCl, 2 mmol/l MgCl₂, 30 mmol/l Mes, pH 6.0 or Hepes pH 7.0 and 25 mmol/l Gly-Gly.

27. The process according to claim 22, wherein said secondary carrier is a biosensor electrode for providing an electrically conductive and solid-type electrode area with at least one electrode, wherein said area is electrically insulated vis-á-vis the measuring solution and vis-á-vis the primary carriers, and said process comprises the step of providing an insulation area in the form of a solids-supported membrane which is built up in layers of a bottom layer of an organic thiocompound as a bottom-most layer facing the electrode and a top layer of an amphiphilic organic compound.

28. The process according to claim 22, wherein said secondary carrier is a biosensor electrode and said process comprises the step of providing an electrode of gold in the electrode area with a monolayer of a long chain alkane thiol as the bottom layer thereon and a monolayer of a lipid as the top layer thereon.

29. The process according to claim 22, wherein said secondary carrier is a biosensor electrode and said process comprises the step of forming the area of the insulation area which covers an electrode of the biosensor electrode as the secondary carrier as a membrane structure in a form selected from the group consisting of a solid-supported double layer membrane and a bi-layer membrane.

30. The process according to claim 22, wherein said primary carrier is selected from the group consisting of a eukaryotic cell, a prokaryotic cell, an oocyte, a bacterium, a virus, an organelle and components thereof and are used in at least one of the native form and in an altered form.

31. The process according to claim 22, wherein said plurality of primary carriers is selected from the group consisting of a vesicle, a liposome and a micellar structure.

32. The process according to claim 22, wherein said type of PepT1 protein is based on the PepT1 protein which originates from a tissue of a mammal selected from the group consisting of the small intestine, kidney, bile duct and pancreas, or wherein said protein is genetically derived from said mammal tissue.

33. The process according to claim 22, wherein said type of PepT1 protein originates from an organism selected from the group consisting of pig, mouse, sheep and man, or is genetically derived from said organism.

34. The process according to claim 22, comprising the step of forming or using the type of PepT1 protein at least partially by stretching through a membrane in the plurality of primary carriers.

35. The process according to claim 22, wherein, as an electrical action, at least one of an electrical current produced by the active site complex or a part thereof or an electrical potential produced thereby is used which is produced respectively by at least one process selected from the group consisting of charge transport, substance transport, charge transfer, substance transfer, conformation changes, ligand binding, ligand addition, ligand release and a combination said processes.

36. The process according to claim 22, wherein said enzymatic property is at least one process selected from the group consisting of:
the bond, attachment or release of the active principle complex or a part thereof or the measuring solution or a part thereof;
the transport or transfer of the active principle complex or a part thereof or the measuring solution or a part thereof;
the chemical conversion or reaction of the active principle complex or a part thereof or the measuring solution or a part thereof;
a conformation change or movement of the active site complex or a part thereof; and
any combination of these processes.

37. The process according to claim 22, wherein said measuring solution is selected from the group consisting of an aqueous measuring solution and an aqueous electrolyte solution.

38. The process according to claim 22, further comprising at least one of the following additional steps:
admixing or injecting the active principle complex, a part or a preform thereof;
replacing the measuring solution or a part thereof; and
chemically converting, physically converting or reacting the measuring solution or a part thereof or of the active principle complex, a part or a preform thereof,
wherein said additional steps carry out at least one of said process steps (c) and (d).

39. The process according to claim 22, further comprising the step of providing a sensor arrangement of biosensor electrode as the secondary carrier and primary carrier attached to said secondary carrier has the measuring solution streaming around or to it in a device selected from the group consisting of a measuring chamber, a measuring area and a measuring vessel.

40. The process according to claim 22, comprising the step of sequentially performing a multiplicity of tests by sequentially replacing the measuring solution.

41. The process according to claim 23, further comprising a washing step between said steps (f) and (g), said washing step comprising introducing the secondary carrier with the plurality of primary carriers into a washing solution.

42. The process according to claim 23, wherein said step (f) is repeated directly before step (h).

43. The process according to claim 23, wherein
the attachment buffer comprises a suitable cation and has a pH of 6-8;
the non-activating solution comprises at least one suitable cation and has a pH of 6-8; and
the activating solution comprises at least one suitable cation, a substrate of type PepT1 protein in a concentration of approximately >0 to 100 mmol/l and has a pH of 6-8.

44. The process according to claim 23, wherein at least one of the non-activating solution and the activating solution comprises a compensator.

45. A screening process for identifying at least one of:
an unknown active principle, active principle complex, part or derivative thereof;
the presence of an unknown active principle, active principle complex, part or derivative thereof;
the presence of a known active principle, active principle complex, part or derivative thereof;
the concentration of an unknown active principle, active principle complex, part or derivative thereof;
the concentration of a known active principle, active principle complex, part or derivative thereof; or
any desired combination of the above-mentioned values or properties by using a process selected from the group consisting of claim 1 and claim 6, wherein the active principle, the active principle complex, the part or the derivative thereof modifying an enzymatic property of an active site complex which contains a type of a PepT1 protein or a part thereof.

46. Use of the process according to claim 22 for finding inhibitors, partial or temporary inhibitors, activators or modulators with an enzymatic property of an active site complex which contains a type of a PepT1 protein and in particular the human PepT1 protein.

47. An active principle or active principle complex for modifying an enzymatic property of an active site complex which contains a type PepT1 protein or a part thereof which is, will be or has been identified according to a process selected from the group consisting of claim 22 and claim 26.

48. A process for producing a drug comprising the steps of:
identifying at least one of an active principle and an active principle complex which modifies a certain enzymatic property of an active site complex containing a type PepT1 protein, or a part thereof, or a multiplicity of properties in a suitable manner by a process selected from the group consisting of claim 1 and claim 6; and
producing and/or isolating at least one of the active principle, the active principle complex, a part and a derivative thereof.

49. A test kit for carrying out processes according to a process selected from the group consisting of claim 22 and claim 26, said test kit comprising:
at least one primary carrier; and
a measuring area.

50. A screening process for identifying at least one of:
an unknown active principle, active principle complex, a part and/or derivate thereof;
the presence of an unknown active principle, active principle complex, part and/or derivative thereof;
the presence of a known active principle, active principle complex, part and/or derivative thereof;
the concentration of an unknown active principle, active principle complex, part and/or derivative thereof;
the concentration of a known active principle, active principle complex, part and/or derivative thereof;
any desired combination of the above-mentioned values or properties by using a process selected from the group consisting of claim 22 and claim 26 or the test kit according to claim 29, wherein the active principle, the active principle complex, the part or the derivative thereof modifies an enzymatic property of an active site complex which contains a type of a PepT1 protein or a part thereof.

51. Use of the test kit according to claim 49, for finding inhibitors, activators, partial or temporary inhibitors or modulators of an enzymatic property of an active site complex which contains a type of a PepT1 protein.

52. Use of a drug, said drug being produced according to the process according to claim 48, for inhibiting, partially or temporarily inhibiting or modulating an enzymatic property of an active site complex which contains a type of a PepT1 protein or a part thereof.

53. The process according to claim 23, wherein said second or non-activating solution comprises a compensator, further comprises the step of detecting an electrical action according to said process step (e) and said third or activating solution further contains a compensator.

54. The process according to claim 30, wherein said components comprise membrane fragments or composites thereof and are used in at least form selected from the group consisting of a purified form, a microbiologically modified form and a molecular-biologically modified form.

55. The process according to claim 40, comprising the step of washing or rinsing the measuring chamber in between each sequential step of the step of sequentially replacing the measuring solution.

56. The process according to claim 43, wherein the attachment buffer has a pH of approximately 7, the non-activating solution further comprises a compensator and has a pH of approximately 7, and the activating solution further comprises a compensator and has a pH of approximately 7, and wherein said substrate of type PepT1 protein is Gly-Gly in a concentration of approximately >0 to 100 mmol/l.

57. The process according to claim 26, wherein in steps (f) and (g) said second or non-activating solution comprises a compensator, further comprises the step of detecting an electrical action according to said process step (e) and said third or activating solution further contains a compensator.

58. The process according to claim 26, wherein said secondary carrier is a biosensor electrode for providing an electrically conductive and solid-type electrode area with at least one electrode, wherein area is electrically insulated vis-á-vis the measuring solution and vis-á-vis the primary carriers, and said process comprises the step of providing an insulation area in the form of a solids-supported membrane which is built up in layers of a bottom layer of an organic thiocompound as a bottom-most layer facing the electrode and a top layer of an amphiphilic organic compound.

59. The process according to claim 26, wherein said secondary carrier is a biosensor electrode and said process comprises the step of providing an electrode of gold in the electrode area with a monolayer of a long chain alkane thiol as the bottom layer thereon and a monolayer of a lipid as the top layer thereon.

60. The process according to claim 26, wherein said secondary carrier is a biosensor electrode and said process comprises the step of forming the area of the insulation area which covers an electrode of the biosensor electrode as the secondary carrier as a membrane structure in a form selected from the group consisting of a solid-supported double layer membrane and a bi-layer membrane.

61. The process according to claim 26, wherein said primary carrier is selected from the group consisting of a eukaryotic cell, a prokaryotic cell, an oocyte, a bacterium, a virus, an organelle and components thereof-and are used in at least one of the native form and in an altered form.

62. The process according to claim 61, wherein said components comprise membrane fragments or composites thereof and are used in at least form selected from the group consisting of a purified form, a microbiologically modified form and a molecular-biologically modified form.

63. The process according to claim 26, wherein said plurality of primary carriers is selected from the group consisting of a vesicle, a liposome and a micellar structure.

64. The process according to claim 26, wherein said type of PepT1 protein is based on the PepT1 protein which originates from a tissue of a mammal selected from the group consisting of the small intestine, kidney, bile duct and pancreas, or wherein said protein is genetically derived from said mammal tissue.

65. The process according to claim 26, wherein said type of PepT1 protein originates from an organism selected from the group consisting of pig, mouse, sheep and man, or is genetically derived from said organism.

66. The process according to claim 26, comprising the step of forming or using the type of PepT1 protein at least partially by stretching through a membrane in the plurality of primary carriers.

67. The process according to claim 26, wherein as an electrical action, at least one of an electrical current produced by the active site complex or a part thereof or an electrical potential produced thereby is used which is produced respectively by at least one process selected from the group consisting of charge transport, substance transport, charge transfer, substance transfer, conformation changes, ligand binding, ligand addition, ligand release and a combination said processes.

68. The process according to claim 26, wherein said enzymatic property is at least one process selected from the group consisting of:
the bond, attachment or release of the active principle complex or a part thereof or the measuring solution or a part thereof;
the transport or transfer of the active principle complex or a part thereof or the measuring solution or a part thereof;
the chemical conversion or reaction of the active principle complex or a part thereof or the measuring solution or a part thereof;
a conformation change or movement of the active site complex or a part thereof; and
any combination of these processes.

69. The process according to claim 26, wherein said measuring solution is selected from the group consisting of an aqueous measuring solution and an aqueous electrolyte solution.

70. The process according to claim 26, further comprising at least one of the following additional steps:
admixing or injecting the active principle complex, a part or a preform thereof;
replacing the measuring solution or a part thereof; and
chemically converting, physically converting or reacting the measuring solution or a part thereof or of the active principle complex, a part or a preform thereof,
wherein said additional steps carry out at least one of said process steps (c) and (d).

71. The process according to claim 26, further comprising the step of providing a sensor arrangement of biosensor electrode as the secondary carrier and primary carrier attached to said secondary carrier has the measuring solution streaming around or to it in a device selected from the group consisting of a measuring chamber, a measuring area and a measuring vessel.

72. The process according to claim 26, comprising the step of sequentially performing a multiplicity of tests by sequentially replacing the measuring solution.

73. The process according to claim 72, comprising the step of washing or rinsing the measuring chamber in between each sequential step of the step of sequentially replacing the measuring solution.

74. The process according to claim 26, further comprising a washing step between said steps (f) and (g), said washing step comprising introducing the secondary carrier with the plurality of primary carriers into a washing solution.

75. The process according to claim 26, wherein said step (f) is repeated directly before step (h).

76. The process according to claim 26, wherein the attachment buffer comprises a suitable cation and has a pH of 6-8, the non-activating solution comprises at least one suitable cation and has a pH of 6-8, and the activating solution comprises at least one suitable cation, a substrate of type PepT1 protein in a concentration of approximately >0 to 100 mmol/l and has a pH of 6-8.

77. The process according to claim 76, wherein the attachment buffer has a pH of approximately 7, the non-activating solution further comprises a compensator and has a pH of approximately 7, and the activating solution further comprises a compensator and has a pH of approximately 7 and wherein said substrate of type PepT1 protein is Gly-Gly in a concentration of approximately >0 to 100 mmol/l.

78. The process according to claim 26, wherein at least one of the non-activating solution and the activating solution comprises a compensator.

79. Use of the process according to claim 26 for finding inhibitors, partial or temporary inhibitors, activators or modulators with an enzymatic property of an active site complex which contains a type of a PepT1 protein and in particular the human PepT1 protein.

80. The process according to claim 48, further comprising the steps of:
purifying the active principle, active principle complex, part and/or derivative; and
mixing and /or portioning the active principle, active principle complex, part and/or derivative with a pharmaceutically compatible carrier substance.

81. The test kit according to claim 49, further comprising:
a first or attachment buffer, a second or non-activating solution, a third or activating solution for absorbing a potential active principle complex; and
at least one potential active principle complex.

82. The use of the test kit according to claim 51, wherein said type of a PepT1 protein is the human PepT1 protein.

83. The use of a drug according to claim 52, wherein said type of a PepT1 protein or a part thereof is the human PepT1 protein.
